(19) United States
(12) Patent Application Publication
(10) Pub. No.: US 2010/0272633 A1
(43) Pub. Date: Oct. 28, 2010

(54) METHOD FOR PRODUCING ALCOHOL, METHOD FOR PRODUCING HYDROGEN OR SYNTHESIS GAS USING THE METHOD FOR PRODUCING ALCOHOL, AND ALCOHOL

(75) Inventors: Hisashi Yamamoto, Yokohama-shi (JP); Naoyoshi Iwasaki, Hitachinaka-shi (JP); Shigeki Nagamatsu, Mito-shi (JP); Nobuhiro Yamada, Mito-shi (JP); Yukitoshi Fukuda, Tokyo (JP); Hiromichi Hidaka, Hofu-shi (JP); Hidenobu Oda, Hofu-shi (JP)

Correspondence Address: FITZPATRICK CELLA HARPER & SCINTO, 1290 Avenue of the Americas, NEW YORK, NY, 10104-3800, US

(73) Assignees: JGC CORPORATION, Chiyoda-ku, Tokyo (JP); KYOWA HAKKO BIO CO., LTD., Chiyoda-ku, Tokyo (JP); KYOWA HAKKO CHEMICAL CO., LTD., Chuo-ku, Tokyo (JP)

(21) Appl. No.: 12/743,605

(22) PCT Filed: Dec. 11, 2008
(86) PCT No.: PCT/JP2008/072559

§ 371 Date: May 19, 2010

(30) Foreign Application Priority Data
Dec. 14, 2007 (JP) 2007-323321

(51) Int. Cl. C01B 3/22; C07C 29/76; C07C 29/74

(52) U.S. Cl. 423/648.1; 568/917; 568/913; 48/197 R

(57) ABSTRACT

A method for producing an alcohol of the present invention provides an alcohol having the total sulfur content of 10 ppm by weight or less, by subjecting an alcohol having the total sulfur content of 30 ppm by weight or more and the propanols content of 200 ppm to at least one desulfurization treatment selected from at least one of a desulfurization treatment by a reaction process, a desulfurization treatment with a physical adsorbent, and a desulfurization treatment with a chemical adsorbent.

### TECHNICAL FIELD

The present invention relates to a method for producing an alcohol by removing sulfur compounds from an alcohol containing the sulfur compounds, a method for producing hydrogen or synthesis gas using the method for producing an alcohol, and an alcohol obtained by the method for producing an alcohol. More specifically, the invention relates to a method for producing an alcohol by selectively removing the sulfur compounds from an alcohol not meeting the desired quality as the product due to the inclusion of concentrated impurities that are by-produced in the process of producing an alcohol, thereby producing the alcohol utilizable as the raw material of chemical processes including a catalytic reaction or fuel, a method for producing hydrogen or synthesis gas using the method for producing an alcohol, and the alcohol obtained by the method for producing an alcohol.

Priority is claimed on Japanese Patent Application No. 2007-323321; filed Dec. 14, 2007, the content of which is incorporated herein by reference.

### BACKGROUND ART

Alcohols are important basic materials in the chemical industry, and they can be converted into useful chemical products via various reactions.

In the case of an alcohol containing sulfur compounds, usually, the sulfur compounds are separated from the alcohol by distillation and purified to a sufficient quality level for a reaction employing the alcohol as a raw material. The reason for producing the alcohol by purification in such a manner is because a sulfur compound is susceptible to the catalyst poison.

In addition, alcohols can also be used as fuel for an internal combustion engine used by automobiles or the like or other types of fuel. In the case of an alcohol containing the sulfur compounds, when it is burned, sulfurous acid gas is generated. Therefore, if devices that remove sulfur compounds or sulfurous acid gas are not provided to an alcohol combustion unit, sulfurous acid gas will be discharged to the air thereby exerting a harmful bad effect on the environment such as becoming the cause of acid rain. In the case of fuel for use in automobiles or the like, there is a problem of itself becoming the cause of sulfur poisoning of catalyst.

Among alcohols, ethanol can be most efficiently produced according to fermentation processes, and it has been brought to attention as a carbon neutral fuel or chemical raw material.

Alcohol are, generally, produced from a petroleum-based raw material via a chemical reaction or from a biomass-based raw material via fermentation.

Such products produced according to a chemical reaction or fermentation are crude alcohols, and since they contain impurities in addition to the desired alcohol, they are usually purified by distillation.

In the purification by a distillation process, specifically, in the process of producing an alcohol with sufficient quality for at an industrial level (hereinbelow, referred to as “desired quality”), fractions having a lower-boiling point and a higher-boiling point than the alcohol of desired quality are separated to thereby produce the alcohol of desired quality. As is clear from such the process of separation, not all of the alcohol produced in the production process can be collected as ethanol with the desired quality, and low-purity alcohols which had not reached the desired quality do generate as a by-product.

In such alcohols not meeting the desired quality, sulfur compounds are sometimes included. When such alcohols are used as the raw material in a catalytic reaction, there may be a case where the sulfur content is susceptible to the catalyst poison. In addition, when such alcohols are used as fuel for automobiles, sulfur poisoning to catalyst may occur. Moreover, when such alcohols are used as fuel, it may be the source of releasing harmful gases such as sulfurous acid gas. Since the sulfur compounds produced in the production process are concentrated and included in such alcohols not meeting the desired quality, the degree of adverse effects caused by the sulfur compounds is also great, and as a result, the usable range is limited.

In order to produce the alcohols of desired quality in high yield, countermeasures such as (1) reducing the contents of low-boiling point fractions and high-boiling point fractions, (2) using a higher distillation column with more trays and (3) increasing the feeding flow rate in the distillation column are necessary.

However, in the case of (1), there is a problem that, a very small amount of a sulfur compound which is the problem still mixes in the alcohols with desired quality. In addition, in the cases of (2) and (3), there are problems in that the construction cost for the distillation column increases and that the amount of energy required for distillation increases.

A desulfurization is a removal of sulfur compounds included in the desired material by any method. Among the desulfurization methods, the generally used method is a hydrodesulfurization method of desulfurizing particularly an oil fraction such as naphtha, gasoline, kerosene or light oil.

This hydrodesulfurization method is the method converting a sulfur compound included in the desired material to a compound which is mainly to hydrogen sulfide, according to a hydrogenation reaction, and removing the compound by adsorbing to an absorbent.

However, when the same method is applied to an alcohol, oxygen functional groups in alcohol molecules, which are present in excess, preferentially-act on the activity point of hydrodesulfurization catalyst or absorbent, and thus performance of the catalyst or absorbent cannot sufficiently be exhibited. In addition, depending on the catalyst or absorbent to be used, there may be a case where the alcohol itself undergoes reaction, and thus it is difficult to apply the oil-fraction desulfurization method for desulfurizing an alcohol. This problem results from that the alcohols include an oxygen functional group which is not included in oil fractions.

Accordingly, as a support of the catalyst and a forming agent of the absorbent which are to be used when performing a hydrodesulfurization method for petroleum-based raw materials, γ-alumina has been widely used because of its properties that the specific surface area can be increased, high stability can be provided and the like. However, this γ-alumina is highly reactive with the alcohols and converts into light oxygen-containing hydrocarbons or light hydrocarbons such as methane, ethane, ethylene or propane according to the progression of reactions like decomposition, dehydration, dehydrogenation, polymerization or the like, and thus the yield of low-sulfur alcohol which is the desired product decreases. Consequently, it has been difficult that the hydrodesulphurization method applies to an alcohol.

Furthermore, a method of removing a sulfur compound from the alcohols by an adsorption process has been disclosed (for example, refer to Patent Document 1). However, since this method utilizes an expensive substance such as silver ions, it is not a satisfactory method to be carried out industrially from a practical point of view.

Patent Document 1: Pamphlet of International Publication No. 2005/063354

### DISCLOSURE OF THE INVENTION

### Problems to be Solved by the Invention

The present invention has been made in consideration of the above circumstances, and an object of which is to provide a method for producing an alcohol by selectively removing sulfur compounds from an alcohol not meeting the desired quality of the product due to the inclusion of concentrated impurities that are by-produced in the process of producing an alcohol, thereby producing the alcohol utilizable as the raw material of chemical processes including a catalytic reaction or fuel, a method for producing hydrogen or synthesis gas using the method for producing an alcohol, and the alcohol obtained by the method for producing an alcohol.

### Means for Solving the Problems

The method for producing an alcohol of the present invention provides an alcohol having the total sulfur content of 10 ppm by weight or less by subjecting an alcohol having the total sulfur content of 30 ppm by weight or more and the propanols content of 200 ppm by weight or more to at least one desulfurization treatment selected from a desulfurization treatment by a reaction process, a desulfurization treatment with a physical adsorbent, and a desulfurization treatment with a chemical adsorbent to produce.

The desulfurization treatment by a reaction process may be a step wherein the alcohol is contacted with a catalyst in the presence of hydrogen to produce a compound, and the compound is contacted with an adsorbent.

The catalyst may be supported on a support.

The support or the adsorbent, which gives the recovery ratio of 60% or more pure when pure ethanol is contacted with the support or the adsorbent at 370° C. under normal pressure in a conversion reaction, may be used.

The support and the adsorbent, which gives the recovery ratio of 60% or more pure when pure ethanol is contacted with the support or the adsorbent at 370° C. under normal pressure in a conversion reaction, may be used.

The support or the adsorbent which has the γ-alumina content of less than 3% by weight may be used.

The support and the adsorbent which has the γ-alumina content of less than 3% by weight may be used.

Herein, the γ-alumina is the one which are generally used as a catalyst or an active metallic support used in the reaction such as hydrogenation of hydrocarbons like petroleum, desulfurization, demetallation, isomerization, dehydrogenation or dehydration, and having a crystalline morphology of isometric system and a large size with a surface area per unit mass of 100 to 400 m²/g.

As the support, the one comprising at least one selected from a group consisting of silica, titania, activated carbon, magnesia and alumina may be used.

As the catalyst, the one comprising at least one selected from a group consisting of nickel, molybdenum, cobalt, platinum, palladium, ruthenium and rhodium may be used.

As the adsorbent, the one comprising at least one selected from a group consisting of a zinc compound and an iron compound, and having the total these compounds content of 30% by weight or more, may be used.

As the adsorbent, the one comprising at least one selected from a group consisting of silica, titania, magnesia and alumina may be used.

The reaction temperature may be 400° C. or below and the pressure may be 5 MPaG or lower in the desulfurization treatment by a reaction process.

The desulfurization treatment with a chemical adsorbent may be a step of contacting the alcohol with an ion-exchange resin or a solid catalyst.

The alcohol may be a mixed solution thereof with water.

The desulfurization treatment with a physical adsorbent may be a step of contacting the alcohol with at least one selected from activated carbon, activated earth, diatom earth, silica, alumina and zeolite.

The method for producing hydrogen or synthesis gas of the present invention produces hydrogen or synthesis gas by causing a catalytic reforming reaction of the alcohol produced by the method for producing an alcohol of the present invention.

The alcohol of the present invention is an alcohol having the total sulfur content of 10 ppm by weight or less, which is obtained by subjecting an alcohol having the total sulfur content of 30 ppm by weight or more and the propanols content of 200 ppm by weight or more to at least one desulfurization treatment selected from a desulfurization treatment by a reaction process, a desulfurization treatment with a physical adsorbent and a desulfurization treatment with a chemical adsorbent.

In the desulfurization treatment by a reaction process, the alcohol may be an alcohol which is obtained by contacting the alcohol with a catalyst in the presence of hydrogen to produce a compound, and contacting the compound with an adsorbent.

The alcohol may be an alcohol which is obtained by the desulfurization treatment of contacting the alcohol with an ion-exchange resin or a solid catalyst.

The alcohol may be in a state of a mixed solution thereof with water.

The alcohol may be an alcohol which is obtained by the desulfurization treatment of contacting the alcohol with at least one selected from activated carbon, activated earth, diatom earth, silica, alumina and zeolite.

### EFFECTS OF THE INVENTION

According to the method for producing an alcohol of the present invention, an alcohol having the total sulfur content of 30 ppm by weight or more and the propanols content of 200 ppm by weight or more is subjected to at least one desulfurization treatment selected from a desulfurization treatment by a reaction process, a desulfurization treatment with a physical adsorbent, and a desulfurization treatment with a chemical adsorbent. Consequently, an alcohol, which has the total sulfur content of 10 ppm by weight or less and can be used as the raw material of chemical processes including a catalytic reaction or as fuel for automobiles or other types of fuel, can be produced.

According to the alcohol of the present invention, it is obtained by subjecting the alcohol having the total sulfur content of 30 ppm by weight or more and the propanols content of 200 ppm by weight or more to at least one desulfurization treatment selected from a desulfurization treatment by a reaction process, a desulfurization treatment with a physical adsorbent and a desulfurization treatment with a chemical adsorbent, and it has the total sulfur content of 10 ppm by weight or less. Therefore, the alcohol can be used as the raw material of chemical processes including a catalytic reaction or as fuel for automobiles or other types of fuel.

### BRIEF DESCRIPTION OF THE DRAWINGS

FIG. 1 is a graph showing a temporal change in the temperature distribution inside a reactor when ethanol containing almost no sulfur compounds was used.

FIG. 2 is a graph showing a temporal change in the temperature distribution inside a reactor when a sample E-1 shown in Table 1 was used.

FIG. 3 is a graph showing an adhesion amount distribution of sulfur and carbon in the length direction in a catalyst layer after the low-temperature steam reforming test

FIG. 4 is graph showing a temporal change in the temperature distribution inside a reactor when ethanol after desulfurization treatment was used.

### BEST MODE FOR CARRYING OUT THE INVENTION

Best modes of the present invention for the method for producing an alcohol, the method for producing hydrogen or synthesis gas using the method for producing an alcohol, and the alcohol obtained by the method for producing an alcohol will be described.

Herein, the modes are described in detail only to provide an easier understanding of the purpose of the present invention, and are not intended to limit the invention unless otherwise particularly specified.

[Method for Producing an Alcohol]

The method for producing an alcohol of the present invention is the method for producing an alcohol having the total sulfur content of 10 ppm by weight or less by subjecting the alcohol having the total sulfur content of 30 ppm by weight or more and the propanols content of 200 ppm by weight or more to at least one desulfurization treatment selected from a desulfurization treatment by a reaction process, a desulfurization treatment with a physical adsorbent, and a desulfurization treatment with a chemical adsorbent.

As an alcohol for the alcohol in the present invention, lower alcohols having 1 to 4 carbon atom(s) excluding propanols, such as methanol, ethanol, butanol or the like can be exemplified. Among them, ethanol is preferable.

These alcohols are usually purified via a distillation process, but compounds having a boiling point close to that of the desired alcohols are the problem in this case. Among the alcohols, particularly, lower alcohols like ethanol, butanol or the like may occur an azeotrope with water, and thus the azeotropic point with water may be the problem. In the case of producing the alcohols by a fermentation process, since the fermentation product is usually an aqueous solution of alcohols, the azeotropic point with water has to be considered as long as water coexists in the distillation process.

The total sulfur content in the invention refers to a total amount of compounds containing sulfur included in the alcohol, and the total amount of compounds containing sulfur is expressed in terms of the weight percent of sulfur.

The propanols are compounds allied to lower alcohols such as ethanol and butanol, and are often by-produced in the production step together with the desired alcohols. Since the propanols have its boiling point or the azeotropic point with water close to that of the above-mentioned lower alcohols, separation thereof is not easy. The propanols are separated as the fractions with lower-boiling point or higher-boiling point than the alcohols of desired quality but because of difficulty in its separation. Accordingly, the fractions contain quite an amount of the desired alcohols together with mass of the propanols.

Herein, “the propanols” refers to 1-propanol and 2-propanol.

Among the sulfur compounds, there are compounds derived from petroleum-based raw materials and compounds produced in the fermentation process. Among them, those having a boiling point or an azeotropic point with water each of which is close to that of the lower alcohols such as ethanol or butanol have a subject for producing the alcohol of desired quality. As in the case of the propanols, these sulfur compounds are separated as the fractions having a lower-boiling point or higher-boiling point than the alcohol of desired quality. However, as a result, fractions containing a substantial amount of the alcohol and the propanols are separated from the alcohol of desired quality, and a substantial amount of the sulfur compounds is included in the separated fractions.

The low-purity alcohol fraction separated as the lower-boiling point or higher-boiling point fraction from the alcohols of desired quality is the representative example of “alcohol having the total sulfur content of 30 ppm by weight or more and the propanols content of 200 ppm by weight or more” in the present invention.

A large portion of the propanols and the sulfur compounds which are by-produced in the production process are concentrated and included in the alcohols to be used as raw materials in the method for producing an alcohol of the present invention. The raw materials include a large amount of sulfur compounds which act as catalyst poisons or sources of releasing sulfurous acid gas. Therefore, its industrial application is difficult as long as the sulfur content is not removed.

In particular, since the propanols or various sulfur compounds are generated during the fermentation, the above-mentioned alcohols are often obtained from a plant for producing the desired alcohols via fermentation.

The fermentation process is a method of producing an alcohol to be used as the raw material by using a plant raw material like sugar cane, corn, tapioca, cassava, rice, wheat or the like, a waste wood material, waste paper or the like as the raw material, and performing a fermentation process of the raw material.

Examples of the sulfur compounds included in the alcohol include sulfides such as dimethylsulfide, diethylsulfide, ethylmethylsulfide and dibutylsulfide; disulfides such as dimethyldisulfide, diethyldisulfide, ethylmethyldisulfide and dibutyldisulfide; thiocarboxylates such as methyl thioacetate and S-methyl thioacetic acid; aromatic sulfur compounds such as thiophene, methylthiophene and benzthiophene; esters of sulfurous acid, such as dimethyl sulfite, diethyl sulfite and dibutyl sulfite; esters of sulfuric acid, such as dimethyl sulfate, diethyl sulfate and dibutyl sulfate; and the like.

The desulfurization treatment by a reaction process is the method carrying out a certain chemical reaction to convert the sulfur compounds to compounds having different properties from the former compounds, and then removing the compounds by some kind of method. The most generally used method among the desulfurization treatments by a reaction process is the hydrodesulfurization. The hydrodesulfurization is the method converting the sulfur compounds to hydrogen sulfide by a hydrogenation reaction (hydrogen-addition reaction), and being adsorbed these compounds by an adsorbent for removal.

The hydrogenation reaction (hydrogen-addition reaction) is the reaction in which the alcohol containing the sulfur compounds is contacted with a catalyst in the presence of hydrogen. According to the hydrogenation reaction, the sulfur compounds are converted into hydrogen sulfide, and these compounds can be removed by being adsorbed by an adsorbent.

For the method for producing an alcohol of the present invention, it is preferable that the catalyst to be used for the hydrogenation reaction (hydrogen-addition reaction) is supported on a support.

As the support for the catalyst to be used for the hydrogenation reaction, it is preferable that the recovery ratio of pure ethanol is 60% or more when pure ethanol is contacted with the support at 370° C. under normal pressure in a conversion reaction.

In addition, it is preferable that the content of γ-alumina in the support is less than 3% by weight.

As the support, those comprising at least one selected from a group consisting of silica (SiO₂), titania (TiO₂), activated carbon (AC), magnesia (MgO) and α-alumina (α-Al₂O₃) can be exemplified.

As the catalyst to be used for the hydrogenation reaction, those comprising at least one selected from a group consisting of nickel (Ni), molybdenum (Mo), cobalt (Co), platinum (Pt), palladium (Pd), ruthenium (Ru) and rhodium (Rh) can be exemplified. Specific examples thereof include a Co—Mo-based supported oxide catalyst, an Ni—Mo-based supported oxide catalyst, a Pd supported activated carbon catalyst, a Pt supported activated carbon catalyst and the like.

The reaction temperature of the hydrogenation reaction is preferably in the range of 0° C. to 400° C., more preferably in the range of 100° C. to 300° C.

The reason why the reaction temperature of the hydrogenation reaction is preferably in the range of 0° C. to 400° C. is because, at a reaction temperature above 400° C., a decomposition reaction progresses excessively thereby increasing the production amount of light hydrocarbon gas such as methane or ethane, and results in a decrease in yield of low-sulfur-content alcohol which is the desired product, and a decrease in catalytic activity due to the deposition of carbon substances on the catalyst.

In addition, the reaction pressure of the hydrogenation reaction is preferably in the range of normal pressure to 5 MPaG, more preferably in the range of normal pressure to 3 MPaG.

The reason why the reaction pressure of the hydrogenation reaction is preferably in the range of normal pressure to 5 MPaG, at a reaction pressure higher than 5 MPaG, a decomposition reaction progresses excessively thereby increasing the production amount of light hydrocarbon gas such as methane or ethane, and results in a decrease in yield of low-sulfur-content alcohol which is the desired product, and an increase in machinery cost due to a increase in the designed pressure of the reaction apparatus, which thus lowers economic efficiency.

As the adsorbent to be used in the hydrogenation reaction, it is preferable that the recovery ratio of pure ethanol is 60% or more when pure ethanol is contacted with the adsorbent at 370° C. under normal pressure in a conversion reaction.

In addition, it is preferable that the content of γ-alumina in the adsorbent is less than 3% by weight.

As the adsorbent, those comprising at least one selected from a group consisting of a zinc compound such as zinc oxide and an iron compound such as an iron oxide, and having the total content of these compounds at 30% by weight or more can be used.

In addition, as this adsorbent, those comprising at least one selected from silica, titania, magnesia and alumina and having the γ-alumina content of less than 3% by weight can be exemplified.

As the method of contacting the alcohol containing the sulfur compounds with an ion-exchange resin or a solid catalyst, (1) a method which is continuously feeding the alcohol containing the sulfur compounds into a column filled with an ion-exchange resin or a solid catalyst, or (2) a method which is introducing an ion-exchange resin or a solid catalyst together with the alcohol containing the sulfur compounds into a batch-type reactor, thereby contacting with each other under agitating, can be employed.

As the ion-exchange resin, any one of cation-exchange resin and anion-exchange resin or both of them can be used.

As the solid catalyst, activated earth, heteropolyacid, silica, alumina, zeolite or the like can be used.

In the above-mentioned methods (1) and (2), the temperature when contacting the alcohol containing the sulfur compounds with an ion-exchange resin or a solid catalyst (hereinafter, the temperature may also be referred to as “contact temperature”) is preferably in the range of 0° C. to 200° C., more preferably in the range of room temperature (25° C.) to 100° C.

The reason why the contact temperature is preferably to be in the range of 0° C. to 200° C. or below is because, when the contact temperature is within this range, dehydration reaction or condensation reaction of the alcohols due to the catalytic action of the solid catalyst or the ion-exchange resin hardly occurs.

The desulfurization treatment according to the above-mentioned method (1) or (2) may be carried out under pressure depending on the contact temperature.

In addition, a plurality of ion-exchange resins or solid catalysts can be used at a time.

In the desulfurization treatment of contacting the alcohol containing the sulfur compounds with an ion-exchange resin or a solid catalyst, the sulfur compounds included in the alcohol are usually separated from the alcohol by being chemically adsorbed by the ion-exchange resin or the solid catalyst, and the alcohol is desulfurized.

However, since the ion-exchange resin and the solid catalyst have a property which can convert the sulfur compounds into compounds having properties different from the alcohol, the sulfur compounds are separated by a chemical adsorbent. In addition, depending on the compound, the sulfur compounds which had undergone the above-mentioned conversion reaction is separated from the alcohol, and the alcohol is desulfurized.

In addition, since the ion-exchange resin or the solid catalyst may physically adsorb the sulfur compounds, depending on the compound, the alcohol may be desulfurized by a physical adsorbent.

Specifically, the desulfurization treatment contacting the alcohol containing the sulfur compounds with an ion-exchange resin or a solid catalyst is mainly for contacting the alcohol containing the sulfur compounds with an ion-exchange resin or a solid catalyst, and is not necessarily restricted to desulfurization by a chemical adsorbent but desulfurization by a reaction process or desulfurization by a physical adsorbent may be included.

The sulfur compounds converted into the compound having properties different from the alcohol by the above-mentioned treatment are usually separated by a method like distillation, adsorption or the like.

In addition, if the boiling point of the converted sulfur compound is low enough, this sulfur compound can be removed from the system as a gas in the step of contacting with an ion-exchange resin or a solid catalyst. For example, when the sulfur compound is sulfite ester, the sulfite ester converts into sulfurous acid gas by a desulfurization treatment carried out according to the above-mentioned method (1) or (2), and since the sulfurous acid gas has a sufficiently low boiling point, it separates into a gaseous phase in the step of contacting with an ion-exchange resin or a solid catalyst. Herein, a treatment of removing that gaseous phase is necessary to prevent discharge of the sulfurous acid gas to atmosphere.

Furthermore, in the desulfurization treatment by contacting the alcohol containing the sulfur compounds with an ion-exchange resin or a solid catalyst, it is preferable that the desulfurization treatment is applied for a mixed solution prepared in advance by mixing the alcohol containing the sulfur compounds with water. It is assumed that, by using such a mixed solution, some of sulfur compounds react with water and convert into compounds susceptible to desulfurization by a reaction process or compounds that easily separate from the alcohol.

The desulfurization treatment with a physical adsorbent is a method of removing the sulfur compounds by physical adsorption of the sulfur compounds by a suitable adsorbent. As the adsorbent, activated carbon, activated earth, diatom earth, silica, alumina, zeolite or the like can be used.

The desulfurization treatment with a chemical adsorbent is a method of removing the sulfur compounds by chemical adsorption of the sulfur compounds by a suitable adsorbent. As the adsorbent, an ion-exchange resin or those mainly including copper or the like can be used.

For the desulfurization treatment with a physical adsorbent and the desulfurization treatment with a chemical adsorbent, a method continuously feeding the alcohol containing the sulfur compounds into a column filled with the above-mentioned adsorbent or the like can be employed.

In the above-mentioned methods, the temperature when contacting the alcohol containing the sulfur compounds with the adsorbent is preferably in the range of 0° C. to 200° C., and more preferably in the range of room temperature (25° C.) to 100° C.

The reason why the temperature when contacting the alcohol containing the sulfur compounds with the adsorbent is preferably in the range of 0° C. to 200° C. is because, within this temperature range, desorption of the sulfur compound that had been adsorbed by the adsorbent hardly occurs, thereby enhancing the adsorption effect.

With regard to the desulfurization treatment with a physical adsorbent and the desulfurization treatment with a chemical adsorbent, when the adsorbent adsorbs a certain amount of the sulfur compounds, it begins to stop exhibiting its adsorption function properly. In this case, the adsorbent may either be regenerated or replaced with a new adsorbent.

According to the method for producing an alcohol of the present invention, the alcohol having the total sulfur content of 30 ppm by weight or more and the propanols content of 200 ppm by weight or more is subjected to at least one desulfurization treatment selected from a desulfurization treatment by a reaction process, a desulfurization treatment with a physical adsorbent, and a desulfurization treatment with a chemical adsorbent. Accordingly, the alcohol which has the total sulfur content of 10 ppm by weight or less and can be used as the raw material of chemical processes including a catalytic reaction or as fuel for automobiles or other types of fuel can be produced.

### [Method for Producing Hydrogen or Synthesis Gas]

The method for producing hydrogen or synthesis gas of the present invention is the method producing hydrogen or synthesis gas by causing a catalytic reforming reaction of the alcohol obtained by the method for producing an alcohol of the present invention.

Among the methods for producing hydrogen or synthesis gas, the catalytic reforming reaction has made many achievements with petroleum-based raw materials, and the reaction usually includes a low-temperature steam reforming reaction (pre-reforming) and a high-temperature steam reforming reaction.

Herein, the high-temperature steam reforming is a way of producing synthesis gas by mixing hydrocarbon with steam followed by reacting them usually at a high temperature of 800° C. or above and reforming them.

Meanwhile, the low-temperature steam reforming is a reformulation carried out to lessen the load on the reforming reaction at high temperature when various kinds of hydrocarbon species are included, by mixing hydrocarbon with steam in the pre-stage thereby producing a component such as methane from the hydrocarbon species at a temperature between 250° C. and 550° C.

According to the low-temperature steam reforming reaction which is the first step of the catalytic reforming reaction, ethanol is converted into hydrogen or synthesis gas including methane, carbon dioxide, hydrogen, carbon monoxide as the main ingredient. The obtained hydrogen or synthesis gas can be used as a fuel alternative to petroleum.

Accordingly, if the low-temperature steam reforming reaction proceeds without problems, the following high-temperature steam reforming reaction can be easily carried out.

### [Alcohol]

The alcohol of the present invention is an alcohol having the total sulfur content of 10 ppm by weight or less, which is obtained by subjecting an alcohol having the total sulfur content of 30 ppm by weight or more and the propanols content of 200 ppm by weight or more to at least one desulfurization treatment selected from at least one of a desulfurization treatment by a reaction process, a desulfurization treatment with a physical adsorbent and a desulfurization treatment with a chemical adsorbent. That is, the alcohol of the present invention can be obtained by the above-mentioned method for producing an alcohol of the present invention.

Accordingly, the alcohol of the present invention can be used as the raw material of chemical processes including a catalytic reaction or as fuel for automobiles or other types of fuel.

### EXAMPLES

Hereinbelow, the invention will be described in more detail with reference to examples, but the invention is not limited by the examples below.

### Reference Example 1

In Table 1, compositions of the alcohol containing the sulfur compounds and the propanols are exemplified. All of them are the fractions having lower-boiling point separated in the distillation purification step.

TABLE 1

| | | |
|---|---|---|
| Sample | Alcohol | Other Components (wt · ppm) |

| Name | (wt %) | n-propanol | i-propanol | Total Sulfur |
|---|---|---|---|---|
| E-1 | ethanol - | 8600 | 50 | 55 |
| | 93% | | | |
| E-2 | ethanol - | 250 | 15 | 33 |
| | 90% | | | |
| E-3 | ethanol - | 3700 | 33 | 125 |
| | 90% | | | |

The concentration measurement conditions for ethanol, n-propanol and i-propanol are shown below.

As the detector, FID (Hydrogen Flame Ionization Detector) was used.

As the carrier gas, nitrogen gas was used.

The total sulfur content in the alcohols was measured by gas chromatography. The measurement for the total sulfur content is shown below. The measurement results are expressed in ppm by weight of sulfur.

As the gas chromatography, GC-14B (manufactured by Shimadzu Corporation) was used.

As the detector, FPD (Flame Photometric Detector, S filter, manufactured by Shimadzu Corporation) was used.

As the analytical column, β,β-ODPN 25% Uniport HP 60/80 Glass φ3 mm×5 m (manufactured by GL Science Inc.) was used.

The sample introducing temperature was set to 95° C., the column temperature was set to 90° C., and the detection temperature was set to 95° C.

As the carrier gas, helium gas was used.

### Reference Example 2

Influence of the sulfur compound on the steam reforming reaction of ethanol was exemplified.

The low-temperature steam reforming reaction was carried out using a reactor filled with a reforming catalyst (trade name: N-185, manufactured by Nikki Chemical Corporation) which is placed in a sand fluidizing vessel kept at a temperature of 330° C., and at a reactor pressure of 1.5 MPaG, with a water/ethanol ratio of 2.0 mol/mol.

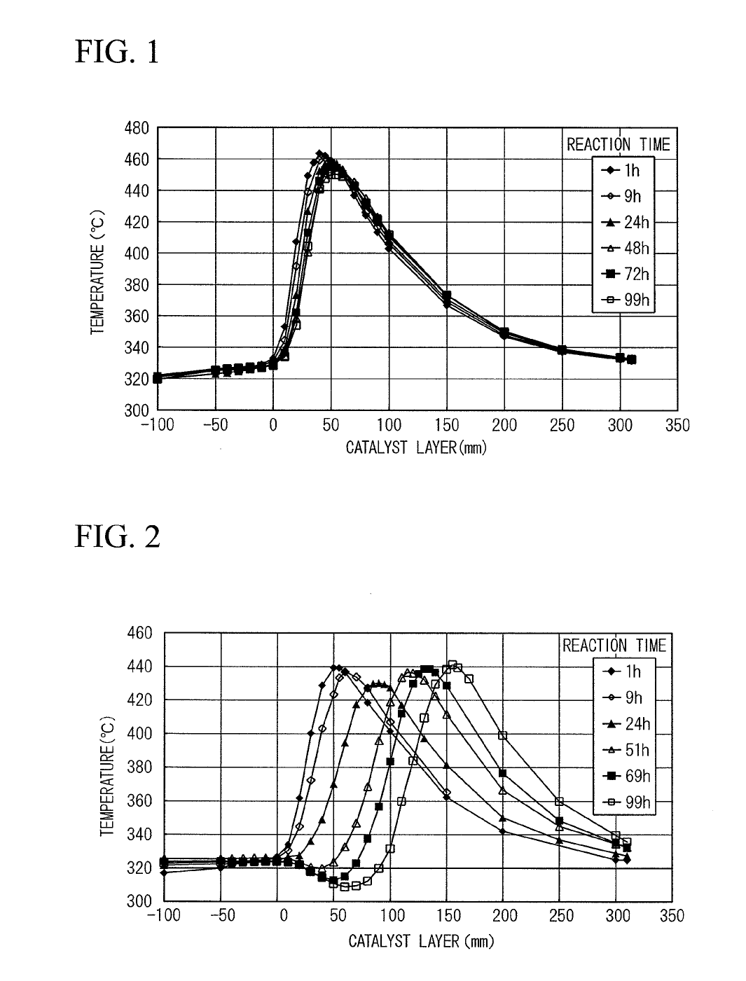

FIG. 1 is a graph showing a temporal change in the temperature distribution inside the reactor when ethanol containing almost no sulfur compounds is used.

FIG. 2 is a graph showing a temporal change in the temperature distribution inside a reactor when a sample E-1 shown in Table 1 is used.

In the low-temperature steam reforming reaction, heat generated as the reaction progresses increased the catalyst temperature, but as shown in FIG. 1, when ethanol contained almost no sulfur compounds, the position of heat generating is almost no change in the length direction of a catalyst layer in the reactor. On the other hand, as shown in FIG. 2, when ethanol contained sulfur compounds, the position of heat generating is transferred to a downstream side in the length direction of a catalyst layer in the reactor.

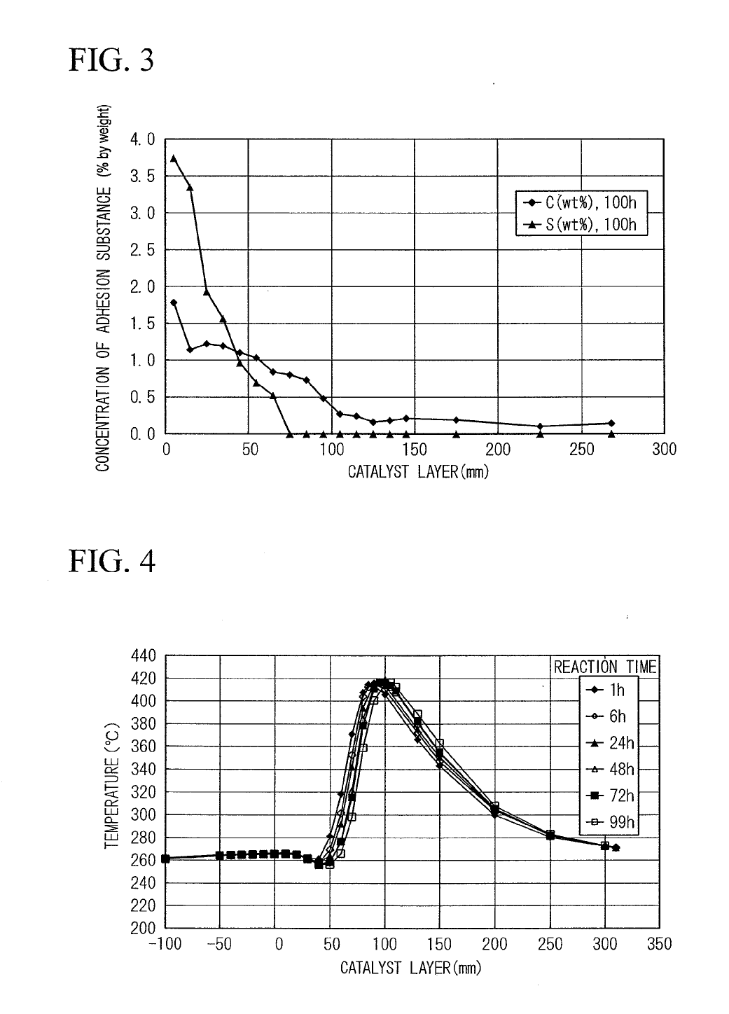

This phenomenon is caused by the sulfur compound poisoning the catalyst, and as shown in FIG. 3, this phenomenon can also be confirmed from the results obtained by the measurement of adhesion amount distribution of sulfur and carbon in the length direction of the catalyst layer after the low-temperature steam reforming reaction test. That is, when a sulfur compound was provided, the low-temperature steam reforming reaction did not proceed due to the gradual adsorption of the sulfur compound from the upstream side of the catalyst layer, and the production of soot caused by a thermal decomposition of ethanol or the like was confirmed. These findings suggest that it is necessary to remove the sulfur compounds in order to treat ethanols as a petroleum-based raw material, by the low-temperature steam reforming reaction.

### [Desulfurization Reaction Test]

For the following desulfurization reaction test, Sample E-1 shown in Table 1 was used as ethanol.

In addition, for the following desulfurization reaction test, a desulfurization reaction was carried out using a reactor connected with a desulfurizing catalyst and an adsorbent which are placed in a sand fluidizing vessel kept at a temperature of 350° C., and at the pressure inside the reactor of 2.0 MPaG, in the presence of hydrogen, and with a hydrogen/ethanol ratio of 0.3 mol/mol.

When a CoO—MoO₃ supported desulfurizing catalyst and a NiO—MoO₃ supported desulfurizing catalyst were used, a sulfurization treatment with hydrogen sulfide was carried out as the pretreatment of those catalysts.

A ZnO adsorbent was prepared by compression-molding a reagent (trade name: zinc oxide KCl class, ZnO purity: 99% by weight or higher, alumina: 0.0%, manufactured by Katayama Chemical Ltd.) and then arranging particles to be between 1.7 mm and 2.8 mm in diameter. This was provided as. “Adsorbent A”. In addition to this, ZnO having different purity and alumina content (ZnO purity: 89.0% by weight, alumina: 4.0% by weight) was used after arranging particles to be between 1.7 mm and 2.8 mm in diameter. This was provided as “Adsorbent B”.

Also, as the iron adsorbent, Fe₂O₃ (Fe₂O₃ purity: 36.0% by weight, alumina: 1.0% by weight) was used after arranging particles to be between 1.7 mm and 2.8 mm in diameter. This was provided as “Adsorbent C”.

### Comparative Example 1

The desulfurization reaction of non-desulfurized ethanol was carried out using CoO—MoO₃/γ-Al₂O₃ (referred to as “Catalyst A”; trade name: CDS-LX1, manufactured by Catalysts and Chemicals Ltd.) as the desulfurizing catalyst and ZnO (“Adsorbent B”) as the adsorbent.

While the total sulfur content of ethanol of Sample E-1 was 55 ppm by weight, in this comparative example, the total sulfur content of ethanol after the desulfurization treatment was 51.4 ppm by weight, showing that only a slight desulfurization reaction had proceeded.

### Comparative Example 2

The reason why only a slight desulfurization reaction proceeded as in Comparative Example 1 was assumed as that the desulfurization reaction was suppressed by an acid point resulted from γ-Al₂O₃ included in the catalyst. Therefore, CoO—MoO₃/SiO₂ (referred to as “Catalyst B”) which is the desulfurizing catalyst employing SiO₂ as a support exhibiting almost no acidic property, and ZnO (Adsorbent B) as the adsorbent were used to carry out the desulfurization reaction of non-desulfurized ethanol.

While the total sulfur content of ethanol of Sample E-1 was 55 ppm by weight in this comparative example, the total sulfur content of ethanol after the desulfurization treatment was 48.7 ppm by weight, showing that only a slight desulfurization reaction had proceeded.

### Comparative Example 3

The reaction was carried out using Sample E-1 shown in Table 1 and the desulfurizing catalyst (Catalyst A) in which molybdenum and cobalt were supported as active metals on a support including γ-alumina which has been used in a hydrodesulfurization of petroleum-based raw materials, at a temperature of 350° C., a reaction pressure of 2.0 MPaG, in the presence of hydrogen, and with a hydrogen/methanol ratio of 0.2 mol/mol. Then, a hydrodesulfurization treatment employing the ZnO-based adsorption catalyst (Adsorbent A) was carried out.

While the total sulfur content of ethanol of Sample E-1 was 55 ppm by weight in this comparative example, the total sulfur content of ethanol after the desulfurization treatment was 50 ppm by weight. This is because, since γ-alumina has an ability to make alcohol undergo a dehydration reaction, ethylene produced from this dehydration reaction of alcohol reacts with hydrogen sulfide produced from the hydrodesulfurization reaction, thereby causing progression of a side reaction that produces ethanethiol or diethylsulfide, and therefore sulfur in the alcohol cannot be adsorbed as the hydrogen sulfide for removal.

### Example 1

Accordingly, the reason why only a slight desulfurization reaction proceeds as in Comparative Examples 1 and 2 was assumed as that the desulfurization reaction was suppressed by the acidic property resulted from γ-Al₂O₃ included in the catalyst and the acidic property resulted from γ-Al₂O₃ included in the adsorbent. Therefore, CoO—MoO₃/SiO₂ (Catalyst B) which is the desulfurizing catalyst employing SiO₂ as a support exhibiting almost no acidic properties, and a γ-Al₂O₃-free ZnO adsorbent (Adsorbent A) were used to carry out the desulfurization reaction of non-desulfurized ethanol.

While the total sulfur content of ethanol of Sample E-1 was 55 ppm by weight, the total sulfur content of ethanol after the desulfurization treatment was 1.5 ppm by weight in this example, showing a remarkable progression of desulfurization reaction. Hereinafter, ethanol of Sample E-1 was used for Examples 2 to 16 as the non-desulfurized ethanol.

From the results of Comparative Examples 1 to 3 and Example 1, it was assumed that to carry out a desulfurization reaction of alcohol with the catalyst containing γ-Al₂O₃ is difficult.

The results of Comparative Examples 1 to 3 and Example 1, and the results of Examples 2 to 6 which will be described later are shown in Table 2.

### Example 2

The desulfurization reaction of non-desulfurized ethanol was carried out using CoO—MoO₃/TiO₂ (referred to as “Catalyst C”) which is the desulfurizing catalyst employing TiO₂ as a support, and a γ-Al₂O₃-free ZnO adsorbent (Adsorbent A).

In this example, the total sulfur content of ethanol after the desulfurization treatment was 3.8 ppm by weight.

### Example 3

The desulfurization reaction of non-desulfurized ethanol was carried out using CoO—MoO₃/AC (referred to as “Catalyst D”) which is the desulfurizing catalyst employing activated carbon (AC) as a support, and a γ-Al₂O₃-free ZnO adsorbent (Adsorbent A).

In this example, the total sulfur content of ethanol after the desulfurization treatment was 1.4 ppm by weight.

### Example 4

The desulfurization reaction of non-desulfurized ethanol was carried out using CoO—MoO₃/MgO (referred to as “Catalyst E”) which is the desulfurizing catalyst employing MgO as a support, and a γ-Al₂O₃-free ZnO adsorbent (Adsorbent A).

In this example, the total sulfur content of ethanol after the desulfurization treatment was 4.7 ppm by weight.

### Example 5

The desulfurization reaction of non-desulfurized ethanol was carried out using CoO—MoO₃/α-Al₂O₃ (referred to as “Catalyst F”) which is the desulfurizing catalyst employing α-Al₂O₃ as a support, and a γ-Al₂O₃-free ZnO adsorbent (Adsorbent A).

In this example, the total sulfur content of ethanol after the desulfurization treatment was 4.5 ppm by weight.

While the total sulfur content of ethanol of Sample E-1 was 55 ppm by weight, total sulfur contents of ethanol after the desulfurization treatment were 5 ppm by weight or less in Examples 1 to 5, thereby showing a remarkable progression of desulfurization reaction. Among them, a combination of CoO—MoO₃/AC (Catalyst D) which is the catalyst employing activated carbon as a support and a γ-Al₂O₃-free ZnO adsorbent (Adsorbent A) performed a high desulfurization performance.

### Example 6

The desulfurization reaction of non-desulfurized ethanol was carried out using NiO—MoO₃/SiO₂ (referred to as “Catalyst G”) which is the desulfurizing catalyst employing SiO₂ as a support and NiO—MoO₃ as the catalytic active metal, and a γ-Al₂O₃-free ZnO adsorbent (Adsorbent A).

While the total sulfur content of ethanol of Sample E-1 was 55 ppm by weight, the total sulfur content of ethanol after the desulfurization treatment was 1.7 ppm by weight in this example, showing a remarkable progression of desulfurization reaction. Accordingly, it was realized that the desulfurization performance does not deteriorate even if the catalytic active metal is replaced from CoO—MoO₃ to NiO—MoO₃.

TABLE 2

| | | | Property of Product | |
|---|---|---|---|---|
| | | | liquid | total sulfur |
| | | | yield | content |
| | Catalyst | Adsorbent | (wt %) | (wt · ppm) |
| Example 1 | CoO—MoO₃/SiO₂ | ZnO | 93 | 1.5 |
| | (Catalyst B) | (Adsorbent A) | | |
| Example 2 | CoO—MoO₃/TiO₂ | ZnO | 92 | 3.8 |
| | (Catalyst C) | (Adsorbent A) | | |
| Example 3 | CoO—MoO₃/AC | ZnO | 97 | 1.4 |
| | (Catalyst D) | (Adsorbent A) | | |
| Example 4 | CoO—MoO₃/MgO | ZnO | 95 | 4.7 |
| | (Catalyst E) | (Adsorbent A) | | |
| Example 5 | CoO—MoO₃/α-Al₂O₃ | ZnO | 93 | 4.5 |
| | (Catalyst F) | (Adsorbent A) | | |
| Example 6 | NiO—MoO₃/SiO₂ | ZnO | 94 | 1.7 |
| | (Catalyst G) | (Adsorbent A) | | |
| Compar- | CoO—MoO₃/γ-Al₂O₃ | ZnO | 90 | 51.4 |
| ative | (Catalyst A) | (Adsorbent B) | | |
| Example 1 | | | | |
| Compar- | CoO—MoO₃/SiO₂ | ZnO | 94 | 48.7 |
| ative | (Catalyst B) | (Adsorbent B) | | |
| Example 2 | | | | |
| Compar- | CoO—MoO₃/γ-Al₂O₃ | ZnO | 91 | 50 |
| ative | (Catalyst A) | (Adsorbent A) | | |
| Example 3 | | | | |

### Example 7

The desulfurization reaction of non-desulfurized ethanol was carried out using Pd/α-Al₂O₃ (referred to as “Catalyst H”) which is the desulfurizing catalyst employing α-Al₂O₃ as a support and Pd as the catalytic active metal, and a γ-Al₂O₃-free ZnO adsorbent (Adsorbent A).

In this example, the total sulfur content of ethanol after the desulfurization treatment was 3.2 ppm by weight.

The results of Example 7, and results of Examples 8 to 14 which will be described later are shown in Table 3.

### Example 8

The desulfurization reaction of non-desulfurized ethanol was carried out using Pd/SiO₂ (referred to as “Catalyst I”) which is the desulfurizing catalyst employing SiO₂ as a support and Pd as the catalytic active metal, and a γ-Al₂O₃-free ZnO adsorbent (Adsorbent A).

In this example, the total sulfur content of ethanol after the desulfurization treatment was 0.9 ppm by weight.

### Example 9

The desulfurization reaction of non-desulfurized ethanol was carried out using Pd/AC (referred to as “Catalyst J”) which is the desulfurizing catalyst employing activated carbon (AC) as a support and Pd as the catalytic active metal, and a γ-Al₂O₃-free ZnO adsorbent (Adsorbent A).

In this example, the total sulfur content of ethanol after the desulfurization treatment was 0.7 ppm by weight.

### Example 10

The desulfurization reaction of non-desulfurized ethanol was carried out using Pd/MgO (referred to as “Catalyst K”) which is the desulfurizing catalyst employing MgO as a support and Pd as the catalytic active metal, and a γ-Al₂O₃-free ZnO adsorbent (Adsorbent A).

In this example, the total sulfur content of ethanol after the desulfurization treatment was 2.1 ppm by weight.

### Example 11

The desulfurization reaction of non-desulfurized ethanol was carried out using Pd/TiO₂ (referred to as “Catalyst L”) which is the desulfurizing catalyst employing TiO₂ as a support and Pd as the catalytic active metal, and a γ-Al₂O₃-free ZnO adsorbent (Adsorbent A).

In this example, the total sulfur content of ethanol after the desulfurization treatment was 2.2 ppm by weight.

While the total sulfur content of ethanol of Sample E-1 was 55 ppm by weight, in Examples 7 to 11, the total sulfur content of ethanol after the desulfurization treatment was 3.5 ppm by weight or less, showing a remarkable progression of desulfurization reaction. Among them, the combination of Pd/SiO₂ (Catalyst I) which is the catalyst employing SiO₂ as a support and a γ-Al₂O₃-free ZnO adsorbent (Adsorbent A), and the combination of Pd/AC (Catalyst J) which is the catalyst employing activated carbon as a support and a γ-Al₂O₃-free ZnO adsorbent (Adsorbent A), performed a high desulfurization performance, and they were within the scope applicable to the steam reforming reaction.

### Example 12

The desulfurization reaction of non-desulfurized ethanol was carried out using Pt/α-Al₂O₃ (referred to as “Catalyst M”) which is the desulfurizing catalyst employing α-Al₂O₃ as a support and Pt as the catalytic active metal, and a γ-Al₂O₃-free ZnO adsorbent (Adsorbent A).

In this example, the total sulfur content of ethanol after the desulfurization treatment was 2.8 ppm by weight.

The results of Example 12 are shown in Table 3.

### Example 13

The desulfurization reaction of non-desulfurized ethanol was carried out using Ru/α-Al₂O₃ (referred to as “Catalyst N”) which is the desulfurizing catalyst employing α-Al₂O₃ as a support and Ru as the catalytic active metal, and a γ-Al₂O₃-free ZnO adsorbent (Adsorbent A).

In this example, the total sulfur content of ethanol after the desulfurization treatment was 3.5 ppm by weight.

The results of Example 13 are shown in Table 3.

### Example 14

The desulfurization reaction of non-desulfurized ethanol was carried out using Rh/α-Al₂O₃ (referred to as “Catalyst O”) which is the desulfurizing catalyst employing α-Al₂O₃ as a support and Rh as the catalytic active metal, and a γ-Al₂O₃-free ZnO adsorbent (Adsorbent A).

In this example, the total sulfur content of ethanol after the desulfurization treatment was 2.2 ppm by weight.

The results of Example 14 are shown in Table 3.

While the total sulfur content of ethanol of Sample E-1 was 55 ppm by weight, in Examples 12 to 14, the total sulfur contents of ethanol after the desulfurization treatment were 3.5 ppm by weight or less, which showing a remarkable progression of desulfurization reaction. Accordingly, it was realized that the desulfurization performance does not deteriorate even if noble metal such as Pt, Ru or Rh is used as the catalytic active metal.

TABLE 3

| | | | Property of Product | |
|---|---|---|---|---|
| | | | liquid | total sulfur |
| | | | yield | content |
| | Catalyst | Adsorbent | (wt %) | (wt · ppm) |
| Example 7 | Pd/α-Al₂O₃ | ZnO | 99 | 3.2 |
| | (Catalyst H) | (Adsorbent A) | | |
| Example 8 | Pd/SiO₂ | ZnO | 95 | 0.9 |
| | (Catalyst I) | (Adsorbent A) | | |
| Example 9 | Pd/AC | ZnO | 98 | 0.7 |
| | (Catalyst J) | (Adsorbent A) | | |
| Example 10 | Pd/MgO | ZnO | 97 | 2.1 |
| | (Catalyst K) | (Adsorbent A) | | |
| Example 11 | Pd/TiO₂ | ZnO | 99 | 2.2 |
| | (Catalyst L) | (Adsorbent A) | | |
| Example 12 | Pt/α-Al₂O₃ | ZnO | 93 | 2.8 |
| | (Catalyst M) | (Adsorbent A) | | |
| Example 13 | Ru/α-Al₂O₃ | ZnO | 95 | 3.5 |
| | (Catalyst N) | (Adsorbent A) | | |
| Example 14 | Rh/α-Al₂O₃ | ZnO | 96 | 2.2 |
| | (Catalyst 0) | (Adsorbent A) | | |

### Example 15

The desulfurization reaction of non-desulfurized ethanol was carried out using CoO—MoO₃/SiO₂ (Catalyst B) which was the desulfurizing catalyst employing SiO₂ as a support, and a Fe₂O₃ adsorbent (Adsorbent C) which slightly contained γ-Al₂O₃.

In this example, the total sulfur content of ethanol after the desulfurization treatment was 1.4 ppm by weight.

The results of Example 15 are shown in Table 4.

### Example 16

The desulfurization reaction of non-desulfurized ethanol was carried out using Pd/α-Al₂O₃ (Catalyst H) which was the desulfurizing catalyst employing α-Al₂O₃ as a support and Pd as a catalytic active metal, and a Fe₂O₃ adsorbent (Adsorbent C) which slightly contained γ-Al₂O₃.

In this example, the total sulfur content of ethanol after the desulfurization treatment was 2.9 ppm by weight.

The results of Example 16 are shown in Table 4.

While the total sulfur content of ethanol of Sample E-1 was 55 ppm by weight, in Examples 15 and 16, the total sulfur content of ethanol after the desulfurization treatment was 3.5 ppm by weight or less, which showing a remarkable progression of desulfurization reaction. Accordingly, it was found that, as in the case of using a γ-Al₂O₃-free ZnO adsorbent (Adsorbent A), high desulfurization performance can still be attained even if a Fe₂O₃ adsorbent (Adsorbent C) which slightly contained γ-Al₂O₃ is used.

TABLE 4

| | | | Property of Product | |
|---|---|---|---|---|
| | | | liquid | total sulfur |
| | | | yield | content |
| | Catalyst | Adsorbent | (wt %) | (wt · ppm) |
| Example 15 | CoO—MoO₃/SiO₂ | Fe₂O₃ | 92 | 1.4 |
| | (Catalyst B) | (Adsorbent C) | | |
| Example 16 | Pd/α-Al₂O₃ | Fe₂O₃ | 99 | 2.9 |
| | (Catalyst H) | (Adsorbent C) | | |
| Example 1 | CoO—MoO₃/SiO₂ | ZnO | 93 | 1.5 |
| | (Catalyst B) | (Adsorbent A) | | |
| Example 7 | Pd/α-Al₂O₃ | ZnO | 99 | 3.2 |
| | (Catalyst H) | (Adsorbent A) | | |

### [Conversion Reaction Test of Pure Ethanol]

For the conversion reaction test of pure ethanol in Comparative Examples 4 and 5 and Examples 17 to 22, reagent ethanol (purity: 99.5%) manufactured by Wako Pure Chemical Industries Ltd. was used, a predetermined amount of a catalyst support or an adsorbent was filled in a reactor provided to an electric furnace kept at a temperature of 370° C., the pressure inside the reactor was set to a normal pressure (0 MPaG), and ethanol serving as the raw material was fed in the manner of LHSV being 2 h⁻¹ so as to carry out the ethanol conversion reaction test.

### Comparative Example 4

When the ZnO adsorbent (Adsorbent B) was used, the recovery ratio of ethanol was 35.9%.

### Comparative Example 5

When γ-alumina was used, the recovery ratio of ethanol was 4.9%.

It was confirmed that when a γ-alumina-containing catalyst support or adsorbent was brought into contact with alcohol, a reaction like decomposition, dehydration, dehydrogenation, polymerization or the like occurs, and conversion into light oxygen-containing hydrocarbons or light hydrocarbons such as methane, ethane, ethylene or propane proceeds, thereby lowering the ethanol recovery ratio.

The results of Comparative Examples 4 and 5 are shown in Table 5.

### Example 17

When SiO₂ was used, the recovery ratio of ethanol was 99.9%.

### Example 18

When α-alumina was used, the recovery ratio of ethanol was 99.7%.

### Example 19

When activated carbon was used, the recovery ratio of ethanol was 97.8%.

### Example 20

When MgO was used, the recovery ratio of ethanol was 96.6%.

### Example 21

When TiO₂ was used, the recovery ratio of ethanol was 62.9%.

### Example 22

When the ZnO adsorbent (Adsorbent A) was used, the recovery ratio of ethanol was 65.7%.

From the above results, it was confirmed that when a catalyst support or an adsorbent which has an ethanol recovery ratio of 60% or higher was used as the catalyst support or the adsorbent for the desulfurization reaction of ethanol, as shown in Examples 1 to 6 and Examples 7 to 14, the liquid yield of the product was high, and the total sulfur content in the product was 10 ppm or less, and thus was suitable as the catalyst support or the adsorbent when desulfurizing the alcohols.

The results of Examples 17 to 22 are shown in Table 5.

### Example 23

A glass column was filled with 30 mL of granular carbon (trade name: Diahope, manufactured by Mitsubishi Chemical Carbon Corporation), and Sample E-2 shown in Table 1 was continuously fed from the top of the column at a rate of 30 mL/hr, thereby successively obtaining a treated liquid from the bottom of the column.

60 mL of ethanol were fed in the column and then a treated liquid was obtained from the bottom of the column, and 120 mL of ethanol were fed in the column and then a treated liquid was obtained from the bottom of the column, and the measurement to determine the total sulfur content in ethanol was carried out according to the same measurement method as in Reference Example 1. As a result, the total sulfur content after the 60 mL-feeding was 6 ppm (desulfurized ratio of 82%) and the total sulfur content after the 120 mL-feeding was 9 ppm (desulfurized ratio of 73%).

### Example 24

A glass column was filled with 30 mL of a cation resin (trade name: Marathon C, manufactured by Dow Chemical Japan Corporation), and Sample E-1 shown in Table 1 was continuously fed from the top of the column under room temperature at a rate of 30 mL/hr, thereby successively obtaining a treated liquid from the bottom of the column.

3 L of ethanol were fed in the column and then a treated liquid was obtained from the bottom of the column, 9 L of ethanol were fed in the column and then a treated liquid was obtained from the bottom of the column, and 18 L of ethanol were fed in the column and then a treated liquid was obtained from the bottom of the column, and a measurement to determine the total sulfur content in ethanol was carried out according to the same measurement method as in Reference Example 1.

The total sulfur content in ethanol before the desulfurization treatment, the total sulfur content in the treated liquid after the 3 L-feeding, the total sulfur content in the treated liquid after the 9 L-feeding, and the total sulfur content in the treated liquid after the 18 L-feeding are shown in Table 6.

### Example 25

The total sulfur content in ethanol before the desulfurization treatment, the total sulfur content in the treated liquid after the 3 L-feeding, the total sulfur content in the treated liquid after the 9 L-feeding, and the total sulfur content in the treated liquid after the 18 L-feeding were measured in the same manner as in Example 18, except that a mixed solution prepared by mixing Sample E-1 shown in Table 1 with pure water so as to be the molar ratio of 1:2 was used. The results are shown in Table 6.

From the results in Table 6, the total sulfur content was reduced to 24 ppm by weight at a point when ethanol was mixed with pure water, and this total sulfur content was further reduced by performing the desulfurization treatment with an ion-exchange resin, and thus ethanol desulfurized to the desired level could be obtained.

### Example 26

The total sulfur content in ethanol before the desulfurization treatment, the total sulfur content in the treated liquid after the 3 L-feeding, the total sulfur content in the treated liquid after the 9 L-feeding, and the total sulfur content in the treated liquid after the 18 L-feeding were measured in the same manner as in Example 18, except that a mixed solution prepared by mixing Sample E-3 shown in Table 1 with pure water to be the molar ratio of 1:2 was used. The results are shown in Table 6.

From the results in Table 6, the total sulfur content was reduced to 39 ppm by weight at a point when ethanol was mixed with pure water, and this total sulfur content was further reduced by performing the desulfurization treatment with an ion-exchange resin, and thus ethanol desulfurized to the desired level could be obtained.

TABLE 5

| | Catalyst Carrier or | Ethanol Recovery |
|---|---|---|
| | Adsorbent | Ratio (%) |
| Comparative | ZnO | 35.9 |
| Example 4 | (Adsorbent B) | |
| Comparative | γ-Al₂O₃ | 4.9 |
| Example 5 | | |
| Example 17 | SiO₂ | 99.9 |
| Example 18 | α-Al₂O₃ | 99.7 |
| Example 19 | AC | 97.8 |
| Example 20 | MgO | 96.6 |
| Example 21 | TiO₂ | 62.9 |
| Example 22 | ZnO | 65.7 |
| | (Adsorbent A) | |

TABLE 6

| | Total Sulfur Content (desulfurized ratio) | | | |
|---|---|---|---|---|
| | Before | After 3L- | After 9L- | After 18L- |
| | treatment | circulation | circulation | circulation |
| | (wt · ppm) | (wt · ppm) | (wt · ppm) | (wt · ppm) |
| Example 24 | 55 | 6.6 (88%) | 6.8 (88%) | 6.7 (88%) |
| Example 25 | 24 | 1.0 (96%) | 0.2 (99%) | 0.2 (99%) |
| Example 26 | 39 | 1.2 (97%) | 0.4 (99%) | 0.4 (99%) |

### Example 27

The low-temperature steam reforming reaction was carried out using the desulfurization-treatment liquid (ethanol) obtained in Example 19, and with the use of a reactor filled with a reforming catalyst which is filled in a sand fluidizing vessel kept at a temperature of 270° C., at the pressure inside the reactor of 1.5 MPaG, in the presence of hydrogen, and with a hydrogen/ethanol ratio of 2.0 mol/mol.

In FIG. 4, a temporal change in the temperature distribution inside the reactor is shown. It shows a moderate change in the temperature distribution of the catalyst layer as compared to the graph shown in FIG. 2, while showing similarity to the graph in FIG. 1. Accordingly, it was confirmed that the low-temperature steam reforming reaction of alcohol can proceeds without causing a reforming catalyst poisoning.

### Example 28

The low-temperature steam reforming reaction was carried out using the desulfurization-treatment liquid (ethanol) obtained in Example 8, under the same reaction conditions as in Example 22. When the temporal change in the temperature distribution inside the reactor was compared with that in a graph shown in FIG. 2, the change in the temperature distribution of the catalyst layer was moderate and showed similarity to the graph in FIG. 1. Accordingly, it was confirmed as that it was possible to perform the low-temperature steam reforming reaction of alcohol without causing a reforming catalyst poisoning.

### INDUSTRIAL APPLICABILITY

According to the method for producing an alcohol of the present invention, the method for producing hydrogen or synthesis gas using the method for producing an alcohol, and the method for producing an alcohol, sulfur compounds can be selectively removed from an alcohol not meeting the desired quality of the product due to the inclusion of concentrated impurities that are by-produced in the production process. Consequently, the alcohol which can be used as the raw material of chemical processes including a catalytic reaction or fuel can be provided.

1. A method for producing an alcohol, wherein
an alcohol having the total sulfur content of 30 ppm by weight or more and the propanols content of 200 ppm by weight or more is subjected to at least one desulfurization treatment selected from a desulfurization treatment by a reaction process, a desulfurization treatment with a physical adsorbent, and a desulfurization treatment with a chemical adsorbent, to produce an alcohol having the total sulfur content of 10 ppm by weight or less.

2. The method for producing an alcohol according to claim 1, wherein
the desulfurization treatment by a reaction process is a step wherein the alcohol is contacted with a catalyst in the presence of hydrogen to produce a compound, and the compound is contacted with an adsorbent.

3. The method for producing an alcohol according to claim 2, wherein the catalyst is supported on a support.

4. The method for producing an alcohol according to claim 2 or 3, wherein
the recovery ratio of pure ethanol is 60% or more when pure ethanol is contacted with the support or the adsorbent at 370° C. under normal pressure in a conversion reaction.

5. The method for producing an alcohol according to claim 3, wherein
the recovery ratio of pure ethanol is 60% or more when pure ethanol is contacted with the support and the adsorbent at 370° C. under normal pressure in a conversion reaction.

6. The method for producing an alcohol according to claim 2 or 3, wherein the content of γ-alumina in the support or the adsorbent is less than 3% by weight.

7. The method for producing an alcohol according to claim 3, wherein the content of γ-alumina in the support and the adsorbent is less than 3% by weight.

8. The method for producing an alcohol according to claim 3, wherein
the support comprises at least one selected from a group consisting of silica, titania, activated carbon, magnesia and alumina.

9. The method for producing an alcohol according to claim 2, wherein
the catalyst comprises at least one selected from a group consisting of nickel, molybdenum, cobalt, platinum, palladium, ruthenium and rhodium.

10. The method for producing an alcohol according to claim 2, wherein
the adsorbent comprises at least one selected from a group consisting of a zinc compound and an iron compound, and
the total content of these compounds is 30% by weight or more.

11. The method for producing an alcohol according to claim 2, wherein
the adsorbent comprises at least one selected from a group consisting of silica, titania, magnesia and alumina.

12. The method for producing an alcohol according to claim 2, wherein
the reaction temperature is 400° C. or below and the pressure is 5 MPaG or lower in the desulfurization treatment by a reaction process.

13. The method for producing an alcohol according to claim 1, wherein
the desulfurization treatment with a chemical adsorbent is a step of contacting the alcohol with an ion-exchange resin or a solid catalyst.

14. The method for producing an alcohol according to claim 13, wherein the alcohol is a mixed solution thereof with water.

15. The method for producing an alcohol according to claim 1, wherein
the desulfurization treatment with a physical adsorbent is a step of contacting the alcohol with at least one selected from activated carbon, activated earth, diatom earth, silica, alumina and zeolite.

16. A method for producing hydrogen or synthesis gas, wherein
hydrogen or synthesis gas is produced by causing a catalytic reforming reaction of the alcohol produced by the method for producing an alcohol according to claim 1.

17. An alcohol having the total sulfur content of 10 ppm by weight or less, which is obtained by subjecting an alcohol having the total sulfur content of 30 ppm by weight or more and the propanols content of 200 ppm by weight or more to at least one desulfurization treatment selected from a desulfurization treatment by a reaction process, a desulfurization treatment with a physical adsorbent and a desulfurization treatment with a chemical adsorbent.

18. The alcohol according to claim 17, which is obtained by the desulfurization treatment by a reaction process in which the alcohol is contacted with a catalyst in the presence of hydrogen to produce a compound, and the compound is contacted with an adsorbent.

19. The alcohol according to claim 17, which is obtained by the desulfurization treatment of contacting the alcohol with an ion-exchange resin or a solid catalyst.

20. The alcohol according to claim 19, wherein the alcohol is in a state of a mixed solution thereof with water.

21. The alcohol according to claim 17, which is obtained by the desulfurization treatment of contacting the alcohol with at least one selected from activated carbon, activated earth, diatom earth, silica, alumina and zeolite.
